(19) United States
(12) Patent Application Publication
(10) Pub. No.: US 2005/0245440 A1
(43) Pub. Date: Nov. 3, 2005

(54) Regulation of a novel colon specific retinol dehydrogenase by apc and cdx2

(75) Inventors: Cicely A. Jette, Jamaica Plain, MA (US); David A. Jones, Salt Lake City, UT (US); Peter W. Peterson, Salt Lake City, UT (US)

Correspondence Address: HELLER EHRMAN WHITE & MCAULIFFE LLP, 1717 RHODE ISLAND AVE, NW, WASHINGTON, DC, 20036-3001, US

(73) Assignee: University of Utah Research Foundation, Salt Lake City, UT (US)

(21) Appl. No.: 10/510,214

(22) PCT Filed: Apr. 7, 2003
(86) PCT No.: PCT/US03/10479

§ 371 Date: Apr. 21, 2005

Related U.S. Application Data
(60) Provisional application No. 60/369,849, filed on Apr. 5, 2002.

(51) Int. Cl. C12Q 1/68; A61K 31/203; A61K 38/17; A61K 31/07

(52) U.S. Cl. 514/12; 514/559; 514/725; 435/6

(57) ABSTRACT

New methods of screening for compounds for treating colon cancer are described. Using microarray expression profiling, molecular evidence was obtained supporting lack of differentiation as the mechanism for development of colon polyps and tumors. An absence of retinoic acid (RA) response genes from neoplastic tissue colon tissues was observed. The absence of RA response genes was paralleled by the lack of expression of retinol dehydrogenase 5 and a novel retinol dehydrogenase homolog, RDHL. RDHL expression is highly restricted to normal colon and the colon specific transcription factors, cdx1 and cdx2 can regulate RDHL. Additionally, re-introduction of APC leads to re-expression of RDHL.

This application claims priority to U.S. Provisional Application Ser. No. 60/369,849 filed Apr. 5, 2002, which is hereby incorporated by reference in its entirety.

### BACKGROUND

The development of colon cancer is closely linked to the normal development process for colon epithelial cells. Normal colon epithelium is organized into crypts where cell colonocyte production, differentiation and turnover occur in topographically distinct regions of proliferation, migration, differentiation and apoptosis. Normal colon crypts show proliferation and differentiation zones within the lower two-thirds of the crypt, a migration zone in the upper third and the surface epithelium where senescent cells are eliminated by apoptosis. The transition of normal colon epithelial cells into a carcinoma may be preceded by the formation of aberrant crypt foci, wherein the crypt proliferation zone expands to encompass the entire crypt (1-5). The expansion of the proliferation zone is thought to result in the formation of a polyp, an intermediate stage in development of a carcinoma. Since polyps appear to result from expansion of the crypt proliferation zone, colonocytes within polyps also show an undifferentiated phenotype (1-5). For example, crypts from colon polyps are severely deficient in mucin producing goblet cells, one of the three predominant, terminally differentiated cell types seen within normal crypts. Currently, little is known about the molecular events in normal crypts that govern differentiation. It is also unclear whether defects in both cell proliferation and cell differentiation are required for neoplasm development within the colon.

The histologic features of colon cancer progression are paralleled by distinct genetic events that initiate and promote tumor formation. An inherited colon cancer predisposition, familial adenomatous polyposis (FAP), results from mutations in a single gene known as adenomatous polyposis coli (APC). This syndrome is characterized by the appearance of hundreds to thousands of colon polyps in affected individuals. The APC gene was discovered by genetic linkage analysis in FAP families and was subsequently cloned through positional cloning strategies by a number of groups (6-15). Mutations in the APC gene appear in early adenomas and in aberrant crypt foci, suggesting very early inactivation of APC in adenoma formation (6,16,17). The multiple intestinal neoplasia (Min) mouse lacks functional APC and serves as a model that supports an early role for APC in adenoma formation (6,18-20). Recent studies also indicate that 85% of sporadic, non-polyposis neoplasms carry mutations within the APC gene (21). Taken together, these observations offer strong support for a causative role for the APC gene in the genesis of most colorectal cancers.

Recent investigations have generated a model describing downstream events controlled by APC. In the current model, APC regulates the activity of a transcriptional pathway that may control colonocyte proliferation (22-28). It does so by regulating the levels of β-catenin, a protein thought initially to function as a link between extracellular adhesion molecules and the cytoskeleton. It appears, however, that β-catenin also regulates transcription through a partnership with TCF-LEF transcription factors (22-28). In cells expressing functional APC, APC acts to repress β-catenin levels through ubiquitin-mediated proteolysis (29-35). Low levels of β-catenin prevent activation of TCF-LEF. In cells harboring mutated APC, β-catenin accumulates. This accumulation allows assembly of β-catenin/TCF-LEF complexes and activation of the transcriptional capabilities of TCF-LEF (22-28). β-catenin/TCF-LEF-dependent transcriptional activation of specific cell cycle regulatory genes, like c-myc and cyclin D1, may underlie the development of colon adenomas and colon carcinomas (22-28). Although APC/β-catenin pathway target genes such as c-myc and cyclin D1 offer mechanistic insights into disregulation of colonocyte proliferation (26,36-39), few of the current APC pathway target genes have easily identifiable roles in cellular differentiation.

Retinoids are a class of small lipid mediators derived from vitamin A that have important roles in vision, cell growth and embryonic development. Roles for retinoids in cell growth and development include supporting cellular differentiation (40,41). All-trans-retinoic acid (RA) and 9-cis-RA elicit changes in gene expression and bring about cell growth arrest and differentiation depending on the target cell (42,43). The biological response to all-trans-RA and 9-cis-RA are mediated through the binding and activation of specific RA receptors, retinoic acid receptors (RARα, RARβ and RARγ) or rexinoid receptors (RXRα, RXRβ and RXRγ) (44,45). These receptors belong to the nuclear hormone receptor superfamily and act, following ligand binding, as direct activators or repressors of gene transcription (44,45).

In addition to the nuclear hormone receptors, retinoid responsiveness within cells is governed by retinoid availability (42,43). For the most part, cells acquire retinoids in the form of retinol, an inactive precursor. Tissues must, therefore, convert retinol into RA in order to activate the network of nuclear receptors required to evoke retinoid transcriptional responses. The enzymes that catalyze these conversions fall into three distinct classes that include the alcohol dehydrogenases (ADH), the short-chain dehydrogenases/reductases (SDR) and the aldehyde dehydrogenases (ALDH) (42,43). ADH and SDR enzymes convert retinol into the aldehyde, retinal (42,43). Further conversion of retinal into RA is carried out by the ALDH enzyme family (42,43). Enzymes in each class have broad substrate specificities and can oxidize or reduce many physiologically important alcohols or aldehydes including ethanol, steroids and retinoids (42,43).

The actions of RA can, in turn, be limited by catabolism via cytochrome P450 enzymes (42,43). Although the biochemistry of these retinoid biosynthetic and metabolic enzymes is emerging, little is known about the regulation of these enzymes within tissues or specific cell types.

### SUMMARY OF THE INVENTION

Accordingly, it is an object of the invention to provide compounds which can treat patients suffering from colon tumors and/or polyps.

It is another object of the invention to provide methods for discovering new drugs to treat patients suffering from colon tumors and/or polyps.

These and other methods are accomplished by reference to the following text.

In a compositional sense, the invention provides an isolated DNA molecule containing an RDHL promoter and vectors containing the same. The invention also includes host cells, which preferably are derived from a vertebrate, e.g. mammals or fish, that contain, are transfected or transformed with a vector or DNA encoding an RDHL promoter. In this regard, the RDHL promoter may be operatively linked to a reporter molecule, e.g., a green fluorescent protein molecule or an antibiotic resistance gene. Also provided herein are kits containing (i) a plurality of host cells harboring an RDHL promoter of the invention, (ii) one or more test molecules and (iii) instructions for use.

The invention also includes compositions for treating a patient suffering from a colon tumor or polyps. These compounds contain an effective amount of a retinoid receptor agonist and a permissive factor therefor. A retinoid receptor agonist includes, but is not limited to, retinoic acid or a derivative thereof. By “derivatives” is meant a compound derivative in the form of an ester, amide or the like. As used herein, a “permissive factor” of a retinoid receptor agonist is a molecule or compound that causes or stimulates the activity of the retinoid receptor agonist. The permissive factor can be, e.g. a cdx2 molecule.

The invention also provides methods for treating a patient suffering from a colon tumor. According to one method, the patient is administered an effective amount of a composition containing a retinoid receptor agonist and a permissive factor therefor. Thereafter, (i) a decreased size in the colon tumor in the patient, or (ii) a lack of increase in size of the colon tumor is observed in the patient after a predetermined amount of time, e.g., after multiple dosages over a period of one or more weeks or months. In preferred methods of the invention, a the retinoid receptor agonist (and, at times, the permissive factor) within the composition stimulates the expression of a gene selected from the group consisting of RDHL, CEACAM-1 and CEACAM-5 in the patient.

The invention also provides methods for treating a patient suffering from colon polyps. According to this method, the patient is administered an effective amount of a composition containing a retinoid receptor agonist and, optionally, a permissive factor therefor. Thereafter, (i) a decreased size in the amount of polyps in the patient, or (ii) a lack of increase in the amount of polyps is observed in the patient after predetermined amount of time, e.g., after multiple dosages over a period of one or more weeks or months. In preferred methods of the invention, a composition stimulates the expression of a gene selected from the group consisting of RDHL, CEACAM-1 and CEACAM-5 in the patient.

The invention also provides methods for preventing the progression of disease, i.e. from colon polyp to colon tumor or from a non-polyp to a colon polyp state. This method includes the steps of determining whether a patient is predisposed to tumor or polyp formation and, if so, administering to the patient an effective amount of a composition as described herein.

The invention additionally provides methods for determining whether a test molecule can be useful in treating a patient suffering from colon polyps and/or a colon tumor.

For example, the invention provides methods for determining whether a test molecule can upregulate RDHL expression in a cell. These methods include the steps of: administering the test molecule to a cell harboring an RDHL promoter, as further described herein; and measuring the level of RDHL enzymatic activity, where an increase in RDHL activity is indicative that the test molecule upregulates RDHL expression. In one embodiment, a retinoid receptor agonist is administered to the cell and the test molecule is a permissive factor therefor. In yet another embodiment, the molecule is not a retinoid receptor agonist.

Still other methods are provided which allow the skilled worker to determine whether a test molecule can upregulate RDHL, CEACAM-1 or CEACAM-5 expression in a cell. These methods include the following steps: administering the test molecule to a cell as described above; and measuring the level of gene expression of said RDHL, CEACAM-1 or CEACAM-5. Here, an increase in gene expression is indicative that the test molecule upregulates RDHL, CEACAM-1 or CEACAM-5. The foregoing measuring step involves determining the level of messenger RNA and/or protein corresponding to RDHL, CEACAM-1 or CEACAM-5, respectively.

Methods for measuring mRNA levels are known in the art. For example, microarray methods can be used, employing commercially available arrays from Affymetrix Inc. (Santa Clara, Calif.). Quantitative PCR (qPCR) employs the co-amplification of a target sequence with serial dilutions of a reference template. By interpolating the product of the target amplification with that a curve derived from the reference dilutions an estimate of the concentration of the target sequence may be made. Quantitative reverse transcription PCR (RTPCR) kits are commercially available from, for example, Applied BioSystems (Foster City, Calif.) and Stratagene (La Jolla, Calif.) See also Kochanowsi, Quantitative PCR Protocols” Humana Press, 1999. For example, total RNA may be reverse transcribed using random hexamers and the TaqMan Reverse Transcription Reagents Kit (Perkin Elmer) following the manufacturer's protocols. The cDNA is amplified using TaqMan PCR master mix containing AmpErase UNG dNTP, AmpliTaq Gold, primers and TaqMan probe according to the manufacture's protocols. The TaqMan probe is target-gene sequence specific and is labeled with a fluorescent reporter (FAM) at the 5′ end and a quencher (e.g. TAMRA) at the 3′ end. Standard curves for both an endogenous control and a target mRNA may be constructed and the comparison of the ratio of CT (threshold cycle number) of target gene to control in treated and untreated cells is determined, allowing quantitation of the amount of starting mRNA. Other methods of measuring mRNA levels are known and may be used in the present invention.

Gene expression may be studied at the protein level using well known methods. Quantitative analysis may be achieved, for example, using ELISA methods employing a pair of antibodies specific to the target protein.

### BRIEF DESCRIPTION OF THE DRAWINGS

Table 1. Twenty-five percent of genes lost in colon tumors are RA response genes. Shown are the 25 most down-regulated genes from colon polyp/tumor vs. normal microarray comparisons. Twenty-five percent of these genes are targets of RA in different tissues. RA response genes are indicated in bold/italic along with the relevant literature citations.

FIG. 1. The expression of RA biosynthetic genes is lost in most colon polyps and tumors. Microarray data are plotted according to polyp (black bars) or tumor (grey bars) samples as fold decrease in gene expression compared to a pool of normal colon samples. For instance, the expression level of RDH5 in sample 1 is 4.5 times higher in the normal colon pool than in the tumor sample. Arbitrarily assigned numbers indicate which samples were analyzed for both RDH5 and RDHL expression.

FIG. 2. RDHL plays a significant role in the colon. A Human Multiple Tissue Northern blot from Clontech (East Palo Alto, Calif.) containing a minimum of 1 μg poly-adenylated RNA was probed with the full length coding sequences for RDHL, RDH5 and β-actin (for loading control). The blot was stripped of all radioactivity between hybridizations. Exposure times for the three hybridizations ranged from 1-3 days in order to emphasize the relative tissue distributions of RDH5 and RDHL.

FIG. 3. CDX transcription factors activate the RDHL promoter. HCT116 cells were transfected with expression vector (CDX1, CDX2 or pCDNA3.1 backbone vector), reporter vector (RDH5:LUC or RDHL:LUC) and normalization vector RSV:Renilla luciferase. Fold induction was calculated by dividing reporter luciferase activities in the presence of cdx1 or cdx2 by luciferase activity from the same reporter in the presence of backbone vector.

FIG. 4. 5-Aza-CdR restores RA responsiveness to HT29 cells. HT9 and HCT116 cells were treated with either 5 μM 5-Aza-CdR or PBS vehicle on day 0. Cells were cultured for 10 days and then treated with either 1 μM RA or ethanol vehicle for twenty-four hours in stripped serum before harvesting. Poly-A RNA was isolated and analyzed by microarray. The indicated comparisons to PBS/ethanol treated cells are displayed as fold induction of gene expression. Also shown are the relevant literature citations demonstrating these genes to be RA responsive.

FIG. 5. CDX2 and RDHL are coincidently expressed after treatment with 5-Aza-CdR and RA. HT29 and HCT116 cells were treated with 5 μM 5-Aza-CdR, or PBS vehicle, and cultured for 6 days. Cells were then treated with either 1 μM RA, or ethanol vehicle, for 6 hours before harvesting. (A) A northern blot was produced using 0.3 μg poly-adenylated RNA from each sample. The same blot was used to probe for RDHL, RDH5 and GAPDH (for loading control) with stripping of all radioactivity between hybridizations. (B) A northern blot was obtained using 0.3 μg poly-adenylated RNA from the indicated treatments in addition to 0.25 μg of poly-A RNA from normal colon (NC) as a positive control. The same blot was used to probe for CDX1, CDX2 and GAPDH (for loading control) with stripping of all radioactivity in between hybridizations. (C) The northern blot in (A) was stripped and re-probed for CDX2 and aligned with the RDHL and GAPDH hybridization from (A) for ease of comparison.

FIG. 6. CDX2 and RA synergistically activate the RDHL promoter in RKO cells. RKO cells were transfected with expression vector (CDX2 or pCDNA3.1 backbone vector), reporter vector (PRL:LUC or RDHL:LUC) and normalization vector RSV:Renilla luciferase. After transfection, cells were treated with either 1 μM RA or ethanol vehicle for 24 hours before harvesting. Fold induction was calculated by dividing reporter luciferase activities in the presence of cdx2, RA, or cdx2+RA by luciferase activity from the same reporter in the presence of backbone vector and ethanol vehicle.

FIG. 7. Re-introduction of APC induces RDHL expression. HT29 APC-inducible and β-galactosidase-inducible cells were treated for twenty-four hours with 100 μM ZnCl₂ or water vehicle before harvesting. A Northern blot was produced using 1.2 μg of poly-A RNA. The same blot was used to probe APC, RDHL and GAPDH (for loading control) with stripping of all radioactivity between hybridizations. Band intensities were measured by phosphorimager, and fold RDHL induction was determined by dividing RDHL values (normalized to GAPDH values) from samples treated with ZnCl₂ to the corresponding samples treated with water. RDHL was induced 3.2-fold in APC-inducible cells and 1.2-fold in β-galactosidase-inducible cells.

FIG. 8. Model for β-catenin independent, APC-induced differentiation. In this model, APC controls proliferation and differentiation by separate pathways. In addition to its well-known role in preventing β-catenin/LEF induced proliferation, we propose that APC independently promotes differentiation of colonocytes possibly through activation of cdx2. This leads to increased RA biosynthesis followed by an RA-mediated program of differentiation.

FIG. 9 shows the RDHL sequence.

FIG. 10 shows the RDH5 sequence.

### DETAILED DESCRIPTION

The present invention is based, in part, on the discovery that RDHL expression is highly restricted to normal (i.e. non-neoplastic) colon tissue and that there exist colon specific transcription factors that can regulate RDHL. As RDHL is absent from colon tumors and polyps, restoration of RDHL activity is an effective treatment for colon cancer. Accordingly, the invention provides the sequence of the regulatory molecules (including the promoter sequence) that govern transcription of RDHL.

Further, the present invention provides methods of treating colon polyps and colon tumors. If the disorder to be treated is a colon polyp, then a treatment regimen includes the step of administering to a patient an effective amount of a retinoid receptor agonist and, optionally, a permissive factor therefor. If the disorder to be treated is a colon tumor, then a treatment regimen includes the step of administering to a patient an effective amount of a retinoid receptor agonist and a permissive factor therefor. As used herein, an “effective amount” of a compound is the amount needed to bring about a desired result, e.g. the activation of the RDHL promoter. A “retinoid receptor agonist” hereby is defined as any compound that interacts directly or indirectly with (and activates) one or more retinoic acid receptors.

In a related aspect, the present invention also provides screening assays that allow for the identification of additional drugs and compounds that activate the RDHL promoter. These assays are, therefore, effective for formulating new and important treatments against colon cancer. According to an assay of the invention, the RDHL promoter is operably linked to a reporter protein, e.g. luciferase or green fluorescent protein, such that activation of transcription produces a detectable signal. Methods of operably linking promoter sequences to reporter proteins are well known in the art. Libraries of potential drugs are then screened for transcriptional activation.

To define the molecular mechanisms governing APC-dependent differentiation of colonocytes, gene expression profiles in colon polyps and tumors were compared to normal colonocytes. It was found that that colon polyps and tumors have a profound deficiency of: 1) retinoic acid response genes and 2) retinoic acid biosynthetic enzymes. Because loss of RA biosynthetic genes may be responsible for the absence of RA response genes in neoplastic colon, the present inventors analyzed the regulatory mechanisms that control the expression of two RA biosynthetic genes, RDH5 and RDHL, that were consistently down-regulated in colon polyps and tumors. It was found that RDHL, but not RDH5, is highly expressed in the colon in comparison to its expression in other tissues implying an important role for RDHL in this tissue. These data indicate that cdx1, cdx2 and APC are involved in regulation of RDHL gene expression. Furthermore, in a model of colonocyte differentiation, the coincident expression of RDHL and cdx2 is restored in HT29 colon cancer cells. It was found that APC controls cdx2-induced differentiation by activating the expression of RA biosynthetic genes and, consequently, an RA-induced program of differentiation. Retinoids play a vital role in differentiation and maintenance of a variety of epithelial tissues (40). The date presented herein suggest they fulfill a comparable role in normal colonocyte function.

Regulation of RDHL Expression by Cdx Transcription Factors

In light of the colon specific expression of RDHL and its absence in colon polyps and tumors, the present inventors sought mechanisms that would account for lack of RDHL expression. Particular attention was given to the possibility that the regulation of RDHL was connected to the APC pathway. Based on the current understanding of APC control of β-catenin levels, the first possibility studied was that β-catenin regulated RDHL expression, and that elevated levels of β-catenin served to repress RDHL expression. To test this possibility, the RDHL promoter was cloned behind a luciferase reporter gene to examine the ability of β-catenin to activate or repress luciferase expression. No evidence that β-catenin had direct or indirect effects on the expression of RDHL was found, however. This indicated that the conventional β-catenin pathway does not regulate RDHL.

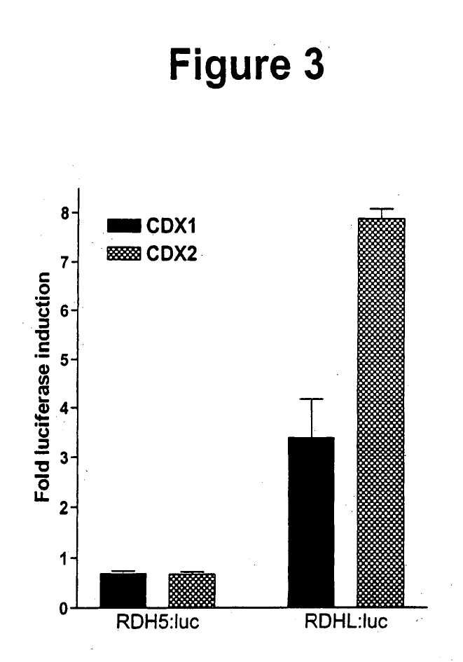

The next possibility was that RDHL was controlled by APC in a pathway separate from β-catenin. In light of the colon-specific expression profile of RDHL, candidate transcription factors that also showed colon specific expression were studied. This led to the cdx transcription factors, cdx1 and cdx2. Cdx1 and cdx2 encode caudal-related homeodomain proteins with important roles in regulating gastrointestinal development in vertebrates (48-51). Their expression is highly specific to colon, and they are absent from human colon tumors (52-54), closely paralleling the regulation of RDHL in colon tissues. To test whether the cdx transcription factors could control RDHL expression, cdx1 and cdx2 were cloned and their ability to activate the RDHL promoter-luciferase construct was studied. FIG. 3 shows the induction of RDHL:LUC activity in HCT116 colon cancer cells co-transfected with cdx1 and cdx2. Both cdx1 and cdx2 induced RDHL:LUC without affecting RDH5:LUC. Consistent with these observations was the presence of three caudal motifs within the promoter of RDHL (data not shown) that conform to other known canonical caudal motifs (55). No such motifs were present in the RDH5 promoter.

RDHL and Cdx2 are Co-regulated in a Model of Colon Tumor Cell Differentiation

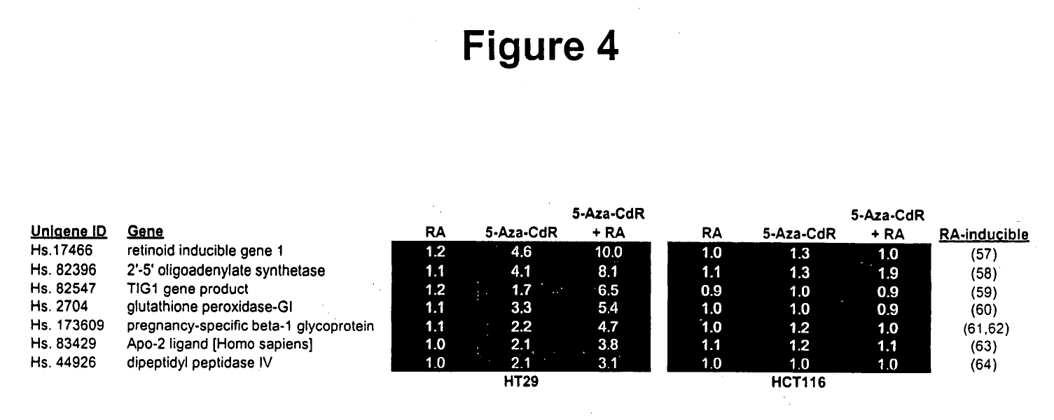

To examine the relationship between colonocyte differentiation and the expression of RDHL, the present inventors examined whether treatment of colon carcinoma cells with 5-aza-2′-deoxycytidine (5-Aza-CdR), a DNA methyltransferase inhibitor, altered RDHL expression. It was found previously that treatment of HT29 cells with 5-Aza-CdR stimulates biological aspects of differentiation and the appearance of differentiation markers (56). In contrast, HCT116 cells show few markers of differentiation following 5-Aza-CdR treatment. The present inventors have now found that treatment of HT29 cells with 5-Aza-CdR alone or in combination with RA enhances RA response gene expression in HT29 cells but not HCT116 cells (FIG. 4). Both cell types were treated with 5-Aza-CdR or PBS vehicle for ten days and then exposed to either RA or ethanol vehicle for 24 hours before harvesting. The samples were compared by microarray and it was found that in HT29 cells, several genes gave a similar expression pattern. Specifically, 1) 5-Aza-CdR alone, but not RA alone, induced the expression these genes, and 2) 5-aza-CdR conferred RA responsiveness to these genes. Out of 65 genes that demonstrated this expression pattern (data not shown), seven are known RA-response genes (57-64) (FIG. 4).

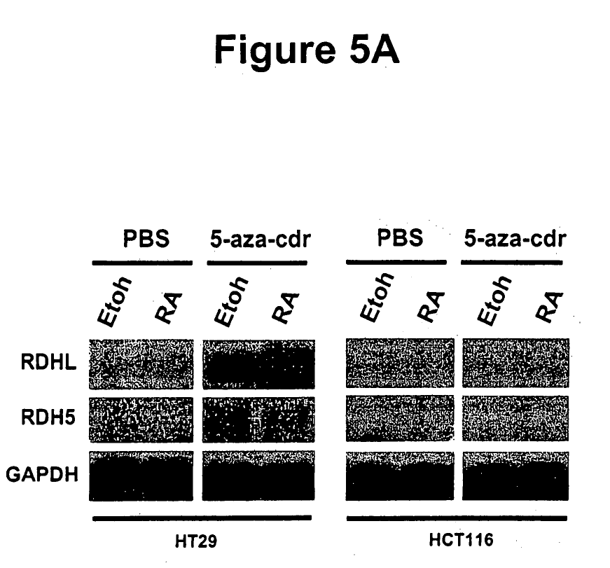

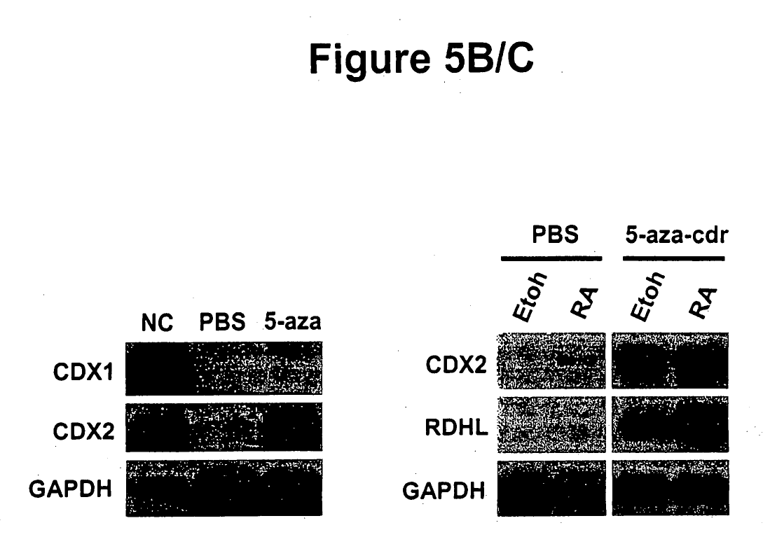

The present inventors examined whether RDHL levels increased in HT29 cells treated with 5-Aza-CdR. Additionally, since RDHL expression has been shown to be activated by RA in airway epithelial cells (47), experiments were carried out to determined whether RA could induce RDHL in colon cells. In this experiment, cells were treated with 5-Aza-CdR or PBS vehicle on day 0 and cultured for six days. Cells were then exposed to RA or ethanol vehicle for six hours. Following treatment, messenger RNAs were harvested and RDHL expression levels were determined by northern analysis (FIG. 5A). Consistent with the above data (FIG. 4), HT29 cells showed expression of RDHL only after treatment with 5-Aza-CdR. Moreover, 5-Aza-CdR treatment enabled induction of RDHL by RA, indicating that RDHL is a RA response gene in colon cells. RDH5 levels were slightly increased in HT29 cells after 5-Aza-CdR treatment but were not affected by RA treatment. As above, no effect of 5-Aza-CdR on RDH5 expression in HCT116 cells was observed. These data indicate that 5-Aza-CdR-induced differentiation of HT29 cells is paralleled by increased RDHL expression, increased RA biosynthesis and increased RA responsiveness.

Experiments using an RDHL:LUC construct indicated that cdxs regulate RDHL. Since 5-Aza-CdR stimulated expression of RDHL in HT29 cells, cdxs also should be induced. Therefore, it was determined whether 5-Aza-CdR changed the levels of cdxs in parallel with RDHL. Northern analyses for cdx2 levels in 5-Aza-CdR treated HT29 cells confirmed that this indeed occurred. FIG. 5B demonstrates the induction of cdx2, but not cdx1, in HT29 cells only after treatment with 5-aza-CdR. Furthermore, expression of cdx2 is coincident with expression of RDHL within the different combinations of 5-aza-CdR and RA treatment (FIG. 5C), which is consistent with a role for cdx2 in regulation of RDHL expression

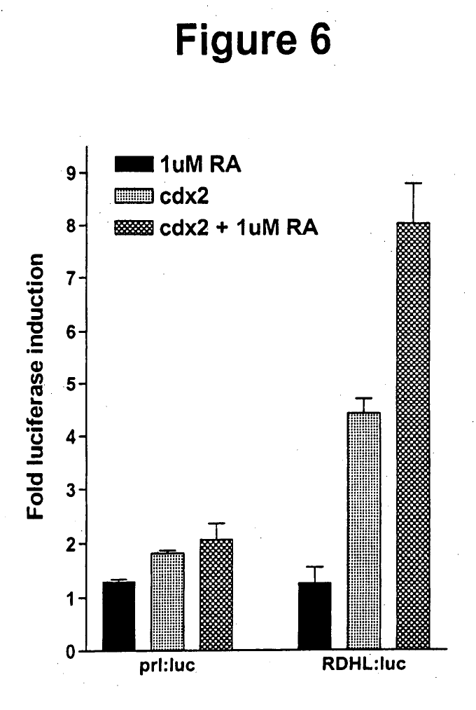

dx2 appeared to be maximally activated by 5-Aza-CdR since additional RA treatment did not increase cdx2 levels (FIG. 5C). That RDHL expression was greatly increased by RA after 5-Aza-CdR treatment (FIG. 5C) suggested that RDHL was further induced by RA independently of an increase in cdx2 expression. In fact, it appeared that cdx2 might be required for RA activation of RDHL in tumor models. To investigate this possibility, the present inventors used transient transfection in RKO colon cancer cells which have wild-type APC and β-catenin, but mutated cdx2. RA treatment alone did not activate the RDHL promoter, but RA in addition to cdx2 activated RDHL:LUC nearly twice as much as cdx2 alone. A luciferase construct driven by −36 to +36 of the prolactin gene shows background activation by cdx2 and RA. Thus, it was found that, similar to 5-Aza-CdR treatment of HT29 cells, exogenous cdx2 must be provided to confer RA responsiveness to the RDHL promoter (FIG. 6).

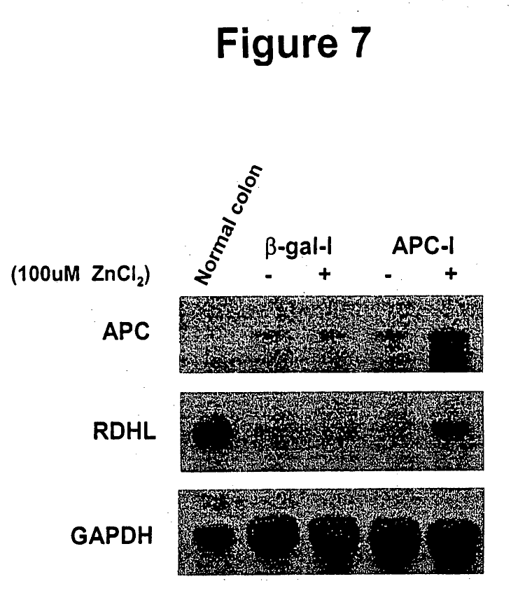

APC has been shown to induce cdx2 expression in HT29 cells ([da Costa, 1999 #154]). This led the present inventors to test whether APC reintroduction would lead to induction of the RDHL gene. HT29 cell line containing a ZnCl₂-inducible APC were constructed nd (described by Morin et et al. [Morin, 1996 #181]. In the absence of ZnCl₂, the APC-inducible cells only express mutant forms of APC. Upon ZnCl₂ addition, WT APC expression is induced. An HT29 cell line containing a ZnCl₂-inducible β-galactosidase construct served as a negative control. Each cell line was treated with 100 μM ZnCl₂ for 24 hours and endogenous gene expression analyzed by Northern analysis. FIG. 7 demonstrates that APC can indeed regulate the expression of RDHL as ZnCl₂ induces a 3.2-fold activation of the RDHL gene only in the APC-inducible cells. Under these experimental conditions, no cdx2 induction after re-introduction of APC was detectable (data not shown).

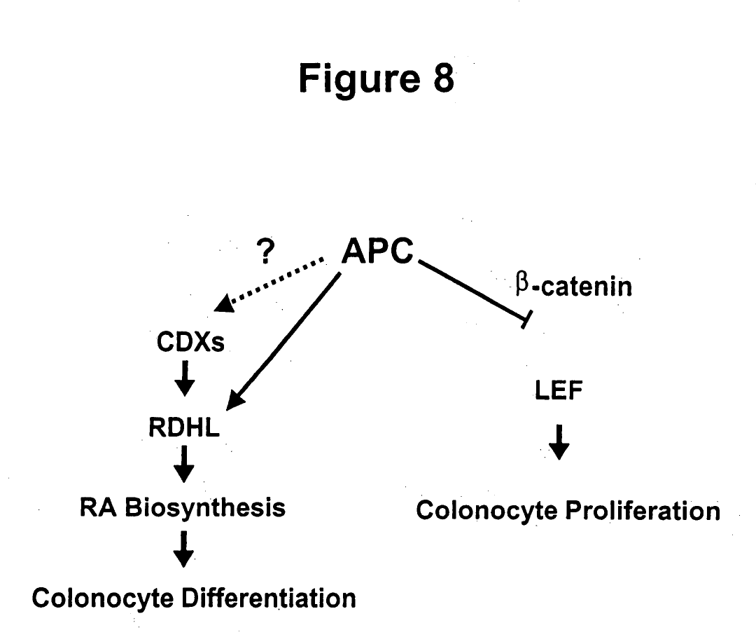

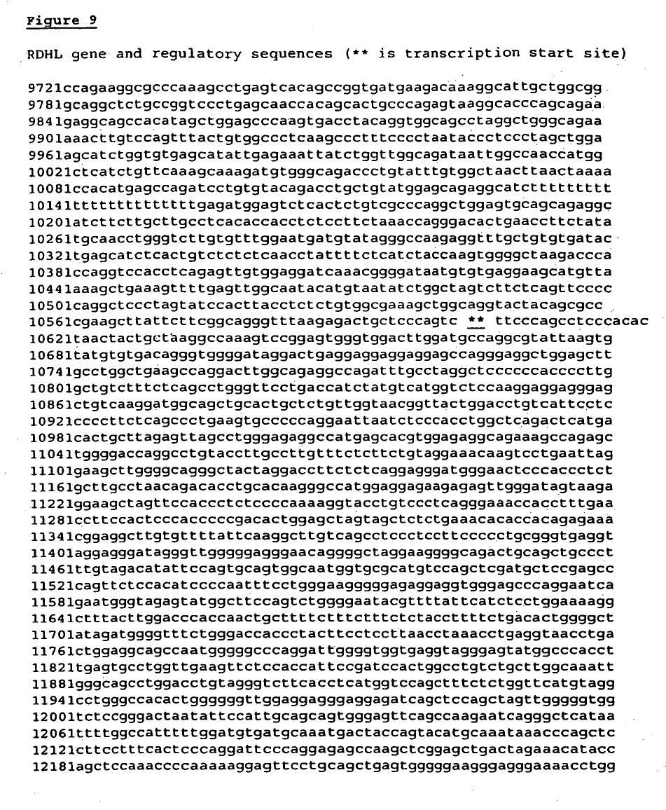

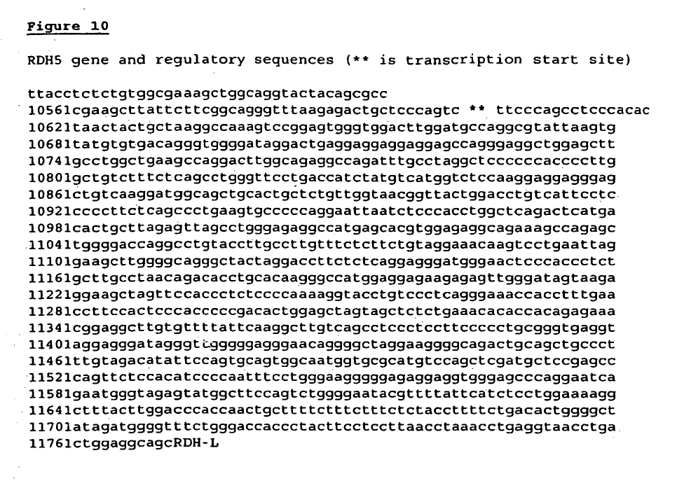

These data led the present inventors to devise the model shown in FIG. 8 proposing that APC simultaneously regulates both proliferation and differentiation. It is well-accepted that APC prevents proliferation by controlling levels of β-catenin, as indicated in the right arm of the model. However, the model proposes that in a β-catenin-independent manner, APC induces RA biosynthesis (possibly through regulation of cdx2) and thus an RA-mediated program of differentiation.

To better define the specific molecular mechanisms that lead to colon cancer, gene expression profiles were compared from colon polyp and tumor to those of normal colon. Difficulties inherent to all microarray studies include the analysis of data and the distillation of the data into a biologically relevant context. Based recent observations (56), a strategy was developed that incorporates a strong biological rationale for understanding complex microarray data. In general, array data for gene sets are examined that reflect the activation state of specific signaling pathways. Using this approach, it was found that a high prevalence of RA response genes among the most down-regulated genes in neoplastic colon (table 1). These genes were categorized as RA-responsive since studies have shown these genes to be RA-inducible in different tissues (65-77). It remains to be determined, however, whether these genes are normally RA responsive in the colon. Nevertheless, the additional absence of the RA biosynthetic genes RDH5 and RDHL from colon polyps and tumors presented a model explaining the lack of RA response genes. Specifically, neoplastic colonocytes may lack the ability to synthesize RA. Lack of RA response genes could reflect this lack of RA biosynthesis.

The existence of biosynthetic and metabolic pathways for retinoids implies that the control of cellular responses to retinoids must, at one level, reside in the control of RA biosynthesis and metabolism. A number of studies in model organisms highlight the importance of RA biosynthesis and metabolism in development and differentiation. For example, deletion of the retinoid metabolizing P450 enzyme CYP26 in mice disrupted anterior-posterior axis development, normal hindbrain patterning, vertebral identity and the development of posterior structures (70,71,72). Similarly, introduction of CYP26 into zebrafish embryos rescued developmental abnormalities resulting from application of excess RA (78). It is clear from these studies that metabolism of RA plays an important role in development and differentiation. It is plausible, therefore, that the balance of retinoid biosynthesis and metabolism plays an important role in colonocyte differentiation. Support for this idea in other tissues comes from recent studies showing that certain breast cancer cell lines failed to synthesize RA (81). Moreover, re-introduction of ALDH6, a gene that converts retinal to RA, restored the ability of MCF-7 breast cancer cells to synthesize RA (82). Finally, retSDR1 and LRAT, two genes in volved in retinol storage, were found to be lost in neuroblastoma and prostate cancer respectively.

Two separate studies have biochemically characterized the enzymatic activity of RDHL. Soref et al demonstrated that RDHL (referred to as hRDH-TBE in their publication) increased the ability of tracheobronchial epithelial cells to convert retinol to RA (47), while Chetyrkin et al determined that RDHL (referred to as 3α-HSD in their publication) prefers different substrates in vitro (83). Specifically, Chetyrkin et al found that RDHL was 100 times more efficient as a 3α-hydroxysteroid-dehydrogenase than as a retinol dehydrogenase. Their in vitro studies demonstrated that RDHL can catalyze the conversion of 3-tetrahydroprogesterone (allopregnanolone) to dihydroprogesterone and 3α-androstanediol to the potent androgen, dihydrotestosterone. Presently, it remains uncertain as to the prevalent enzymatic activity of RDHL in colonocytes. Nevertheless, one might infer critical functions of RDHL by considering the effects of its characterized enzymatic products on colon cancer cells. Studies have shown that retinoids induce markers of differentiation, inhibit cell growth, increase cell adhesion, reduce colony formation, block anchorage-independent growth and suppress invasiveness in colon cancer cells (63,66,76,77). Conversely, while one study showed that treatment of colon cancer cells with either testosterone or progesterone failed to affect cell growth (86), another group found that testosterone may even be associated with increased cell growth (87). Cells undergoing neoplastic changes accumulate mutations that facilitate their growth and survival. Loss of a gene, like RDHL, during colon tumorigenesis often indicates that the gene product serves as an obstacle to neoplastic progression. Given that RA is the only known product of RDHL to abrogate survival of colon cancer cells, it is conceivable that the retinol dehydrogenase activity of RDHL plays a significant role in ensuring the normal development of colonocytes.

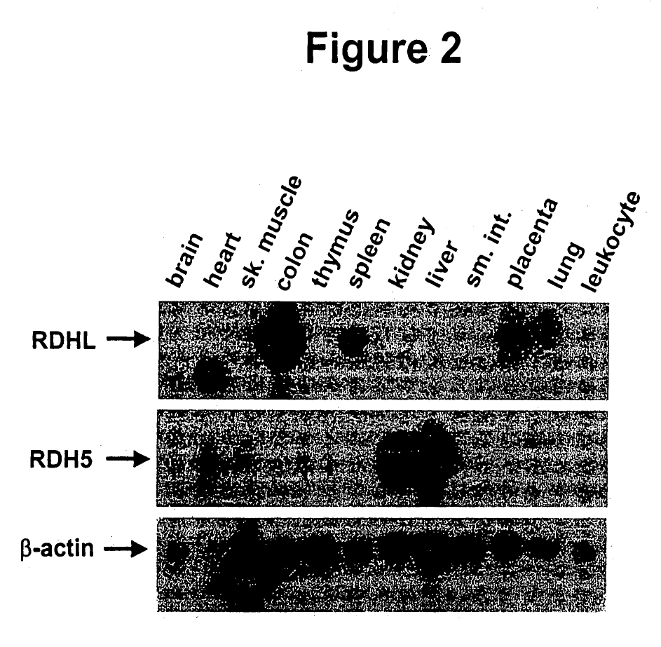

The present inventors have described the loss of two RA biosynthetic genes in colon cancer. Other RA biosynthetic enzymes, including both retinol and retinal dehydrogenases, have been characterized [reviewed in (88)], but it remains unclear as to which play an important role in the colon. It was observed that, relative to its expression level in other tissues, RDHL demonstrated a striking specificity to colon tissue (FIG. 2). High expression of RDHL in the colon has also been observed by two other groups (47,83). Significantly, the distinct tissue expression profiles of RDH5 and RDHL (FIG. 2) indicates that RDHL possibly accounts for a significant proportion of retinol dehydrogenase activity in the colon.

In light of the colon-specific expression of RDHL and its absence in colon polyps and tumors, the present inventors considered mechanisms that might account for lack of RDHL expression. Particular attention was paid to the possibility that the regulation of RDHL was connected to the APC pathway for three reasons. First, RDHL was absent in colon polyps indicating its loss as an early event in tumor progression, like APC. Second, RDHL was absent in approximately 70% of the neoplastic tissue examined. This number is similar to the percentage of tumors harboring mutations in APC. Finally, retinoid production presented an explicit mechanism that may explain how APC promotes normal colonocyte differentiation. β-catenin had no effect on the activation state of RDH5 and RDHL promoters (data not shown), indicating that APC may regulate these genes in a β-catenin-independent manner. Support for a β-catenin-independent arm of the APC pathway arises from the notion that in many systems, cell proliferation and differentiation are counter-regulated. For example, differentiation of hematopoietic tissues requires a molecular switch through which the activity and levels of the proliferative transcription factor, c-myc, are balanced and overcome by the differentiating transcription factor, mad (89-93). Furthermore, in neuronal cells, the retinoblastoma protein is thought to cause cell cycle arrest through transcriptional repression while it independently promotes differentiation through transcriptional activation (94). Therefore, it is conceivable that APC simultaneously regulates both proliferation and differentiation by independent pathways.

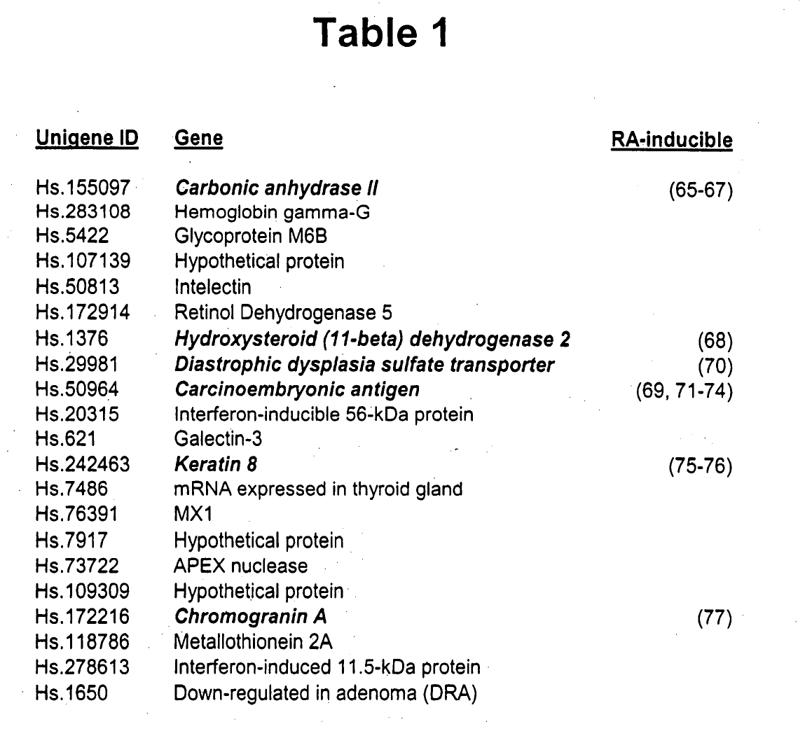

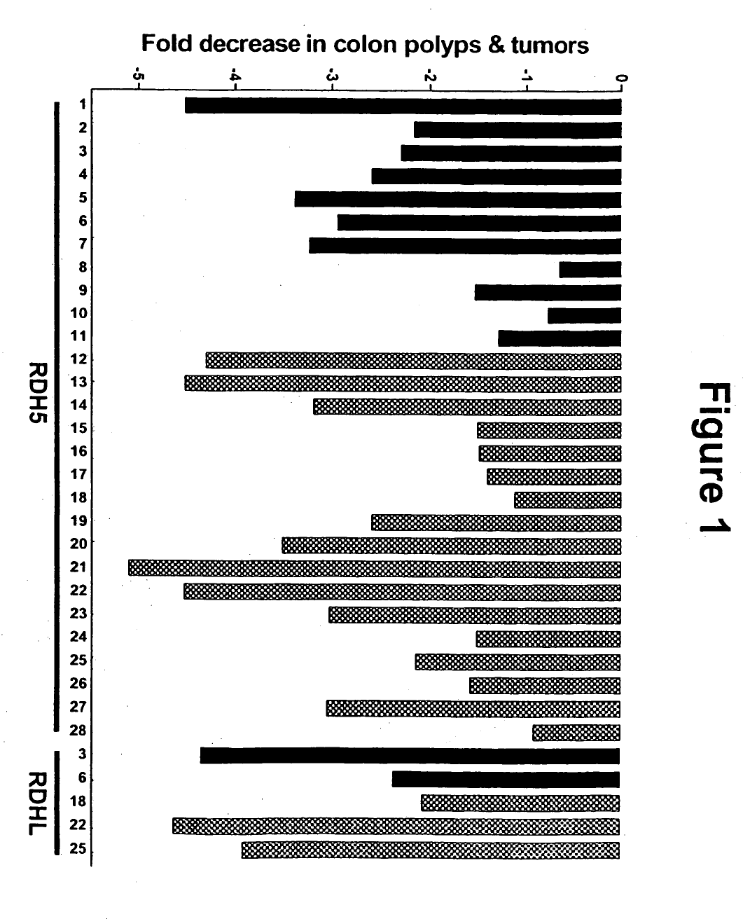

Cdx1 and cdx2 were likely candidates for regulation of RDHL for the following reasons: 1) their expression is specific to the colon, like RDHL [(47,83), FIG. 2], 2) they are lost in colon adenocarcinomas (52-54), like RDHL (FIG. 1), 3) they have been shown to promote differentiation in colon cells (53,54), like RA (71,74,84,85), and 4) with respect to a pro-differentiation arm of the APC pathway, cdx2 was shown to be induced by APC in HT29 cells (46). Consistent with expectations, it was found that not only can both cdxs activate the RDHL promoter (FIG. 3), but both cdx2 and RDHL were coincidently expressed in a model of colonocyte differentiation (FIG. 5C). Additionally, it was found that treatment of HT29 cells with 5-Aza-CdR was similar to overexpression of cdx2 by transient transfection since both confer RA responsiveness to the RDHL promoter (FIGS. 5C and 6). Altogether, these data are consistent with a model whereby cdx2 activates endogenous RDHL expression in colonocytes and offers a molecular link between APC and RDHL expression.

To directly test whether APC regulates the RDHL gene, expression of wild-type APC in HT29 cells was induced. Induction of APC led to the re-expression of RDHL. However, it was not possible to confirm the previous observation that cdx2 expression is induced by APC ([da Costa, 1999 #154]). It is possible that cdx2 induction by APC was below the level of detection. That APC may not regulate the cdx2 gene is suggested by the finding that loss of cdx2 protein expression does not appear to be correlated with loss of APC [Hinoi, 2001 #227]). No studies have addressed whether endogenous cdx2 functions normally when it is expressed in colon tumors.

Few studies have revealed the regulatory mechanisms that control expression of RA biosynthetic genes in any tissues. The loss of RDH5 and RDHL expression in colon cancer emphasizes the importance of understanding these regulatory mechanisms. The findings herein demonstrate that the loss of APC in most colon cancers accounts for the similar loss of RDHL. Both APC and cdx2 have critical roles not only during colonocyte differentiation in the adult (95-98, APC refs), but also during embryonic development (95,99, APC refs). That APC and cdx2 regulate RDHL expression implies that RDHL has similar importance. Furthermore, the observation that RA activates cdx2 (FIG. 5C) implies the existence of a positive feedback mechanism that may serve to amplify developmental signals in the early embryo as well as differentiation signals in adult colonocytes.

An interesting observation arose from treatment of HT29 cells with 5-Aza-CdR in that the drug appeared to make the cells more RA responsive. Previously, the inventors found that 5-Aza-CdR treatment of colon cancer cells induces the expression of several genes (56). As shown in FIG. 4, a subset of these genes are known RA response genes. However, these genes were not induced by 5-Aza-CdR or RA in HCT116 cells (FIG. 4). This raised two main questions from the HT29 data: 1) Why, in the absence of exogenous RA, does 5-Aza-CdR alone induce the expression of RA response genes, and 2) Why are these RA response genes only responsive to RA after 5-Aza-CdR treatment? To answer the first question requires consideration of how cells normally synthesize their own supply of RA. Retinol, normally delivered to cells from the blood, is stored in cellular membranes bound to retinol binding protein until presented to a retinol dehydrogenase to begin the two-step process of RA biosynthesis. Perhaps 5-Aza-CdR treatment enables HT29 cells to endogenously synthesize RA from membrane-stored retinol by activating the expression of retinol dehydrogenases. As a result, RA response genes would be induced by 5-Aza-CdR in the absence of exogenous RA. Consistent with this idea, it was found that in HT29 cells, but not HCT116 cells, both RDHL and RDH5 were re-expressed after 5-Aza-CdR treatment (FIG. 5A).

However, restoration of RA biosynthesis cannot be the only RA response pathway defect in the HT29 and HCT116 cells since, in the absence of 5-Aza-CdR, RA response genes are not induced by exogenous RA (FIG. 4). Additional defects might include mutation or aberrant silencing of RA receptors. Treatment with 5-Aza-CdR is thought to induce differentiation through its ability to de-methylate DNA. That 5-Aza-CdR appeared to restore RA responsiveness to HT29 cells (FIG. 4) suggests that RA receptors may be silenced by DNA methylation in these cells. On the other hand, RA receptors are probably targeted for inactivation by a distinct mechanism in HCT116 cells thus highlighting the importance of RA response pathway inactivation during colon tumorigenesis. In conclusion, although the status of RA receptors in primary colon tumors remains unknown, the data herein suggest the impairment of RA receptors in two colon cancer cell lines. Significantly, mutation or aberrant silencing of RA receptors provides an alternative to loss of RA biosynthetic genes during colon tumorigenesis.

Altogether, the findings herein indicate that the RA response pathway is a target for inactivation in colon cancer. The present inventors' data support the silencing of RA biosynthesis as a downstream consequence of APC mutation, but not necessarily a consequence of β-catenin dis-regulation. A new model (diagrammed in FIG. 8) explains the potential relationship between APC, cdx transcription factors, retinoid biosynthesis and differentiation. Specifically, APC may control intracellular levels of RA, and ultimately, an RA-mediated program of differentiation, by a pathway that is distinct from β-catenin. In apparent contrast to this model, however, are two studies suggesting that β-catenin enhances RA activation of RA response genes. Szeto et al showed that the RA response gene Stra6 (of unknown function) is synergistically activated by Wnt-1 and RA (100). They also found Stra6 expression to be up-regulated in colon tumors. Similarly, Easwaran et al showed that RA activation of an RA-responsive reporter is enhanced by ectopic β-catenin expression (101). However, since RA and β-catenin are primarily associated with differentiation and proliferation, respectively, the relevance of these observations to colon cancer remains uncertain. In fact, this cooperation may not play a specific role in colon tumor promotion since the data suggest that, starting in the early stages of tumorigenesis, RA is not synthesized, and as a result, many RA response genes are not expressed. Thus, overexpression of Stra6 in colon tumors may be due to either increased β-catenin signaling alone, or overactivation of a RA-independent pathway.

Although the past decade has seen tremendous advances in our understanding of the genetic and molecular events underlying APC mutation induced colon tumor development, the generation of pharmacological interventions that capitalize on these molecular advances is lagging. It is of central importance to begin translating our understanding of colon tumor molecular genetics into new treatment strategies that will enhance survival rates. A better, molecular definition of the cellular alterations that promote colon polyp and tumor formation would provide new avenues for rational, therapeutic interventions. The work described herein provides several important new insights into the development of colon tumors. For instance, it reveals a specific molecular link between mutations in APC and the lack of cellular differentiation seen in colon tumor development. Although several target genes for APC/β-catenin/TCF-LEF have been described, there have been no reports of specific, pro-differentiation signaling pathways, like retinoids, that are under the direct control of APC. That cdx2 may regulate RA biosynthesis suggests that cdx2 is emerging as a mediator of an APC-regulated network of pro-differentiation signals. This work permits a testable clinical hypothesis aimed at pharmacological restoration of retinoid activity. Namely, supplementation of APC deficient tissues with exogenous retinoids may prevent colon polyp formation.

### REFERENCES

1. Potten et al., Int J Exp Pathol 78: 21943 (1997).
2. Karam et al., Front Biosci 4: D286-98 (1999).
3. Wright et al., Int J Exp Pathol 81: 1-17-43 (2000).
4. Booth et al, J Clin Invest 105: 1493-9 (2000).
5. Clatworthy et al, Mech Dev 101: 3-9 (2001).
6. Levy et al., Cancer Res 54: 5953-8 (1994).
7. Bodmer et al., Nature 328: 614-6 (1987).
8. Groden et al., Cell 66: 589-600 (1991).
9. Groden et al., Am J Hum Genet 52: 263-72 (1993).
10. Joslyn et al., Cell 66: 601-13 (1991).
11. Kinzler et al., Science 251: 1366-70 (1991).
12. Kinzler et al., Science 253: 661-5 (1991).
13. Leppert et al., Science 238: 1411-3 (1987).
14. Nakamura et al., Am J Hum Genet 43: 63844 (1988).
15. Nishisho et al., Science 253: 665-9 (1991).
16. Jen et al., Cancer Res 54: 5523-6 (1994).
17. Smith et al., Cancer Res 54: 5527-30 (1994).
18. Luongo et al., Cancer Res 54: 5947-52 (1994).
19. Luongo et al., Genes Chromosomes Cancer 17: 194-8 (1996).
20. Su et al., Science 256: 668-70 (1992).
21. Ichii et al., Oncogene 8: 2399-405 (1993).
22. Morin et al., Bioessays 21: 1021-30 (1999).
23. Sparks et al., Cancer Res 58: 1130-4 (1998).
24. Morin et al., Science 275: 1787-90 (1997).
25. Korinek et al. Science 275: 1784-7 (1997).
26. He et al., Science 281: 1509-12 (1998).
27. Polakis et al., Adv Exp Med Biol 470: 23-32 (1999).
28. Polakis et al., Curr Opin Genet Dev 9: 15-21 (1999).
29. Chen et al., Genes Dev 9: 1586-97 (1995).
30. Aberle et al., EMBO J. 16: 3797-804 (1992).
31. Orford et al., J Biol Chem 272: 24735-8 (1997).
32. Salomon et al., J Cell Biol 139: 1325-35 (1997).
33. Jiang et al., Nature 391: 493-6 (1998).
34. Hart et al., Curr Biol 9: 207-10 (1999).
35. Winston et al, Genes Dev 13: 270-83 (1999).
36. Tetsu et al. Nature 398: 422-6 (1999).
37. Ahmed et al., Cell 93: 1171-82 (1998).
38. Wingo et al., J Natl Cancer Inst 91: 675-90 (1999).
39. Gat et al., Cell 95: 605-14 (1998).
40. Noy et al., in Retinoids: The Biochemical Basis of Vitamin A and Retinoid Action H. a. B. Nau, W. S., Ed. (Springer-Verlag, Berlin Heidelberg, 1999) pp. 3-24.
41. De Luca et al., Nutr Rev 52: S45-52 (1994).
42. Blaner et al., in Retinoids: The Biochemical Basis of Vitamin A and Retinoid Action H. a. B. Nau, W. S., Ed. (Springer-Verlag, Berlin Heidelberg, 1999) pp. 117-144.
43. Vogel et al., in Retinoids: The Biochemical Basis of Vitamin A and Retinoid Action H. a. B. Nau, W. S., Ed. (Springer-Verlag, Berlin Heidelberg, 1999) pp. 31-84.
44. Pieddrafita et al., in Retinoids: The Biochemical Basis of Vitamin A and Retinoid Action H. a. B. Nau, W. S., Ed. (Springer-Verlag, Berlin Heidelberg, 1999) pp. 153-174.
45. Beard et al., in Retinoids: The Biochemical Basis of Vitamin A and Retinoid Action H. a. B. Nau, W. S., Ed. (Springer-Verlag, Berlin Heidelberg, 1999) pp. 185-208.
46. da Costa et al., Oncogene 18: 50104 (1999).
47. Soref et al., J Biol Chem 276: 24194-202 (2001).
48. Freund et al., Biochem Cell Biol 76: 957-69 (1998).
49. Murakami et al., Cell Mol Biol (Noisy-le-grand) 45: 661-76 (1999).
50. Bai et al., Mol Carcinog 28: 184-8 (2000).
51. Silberg et al., Gastroenterology 119: 961-71 (2000).
52. Ee et al., Am J Pathol 147: 586-92 (1995).
53. Mallo et al., Int J Cancer 74: 35-44 (1997).
54. Mallo et al., J. Biol Chem 273: 14030-6(1998).
55. Suh et al., Mol Cell Biol 14: 7340-51 (1994).
56. Karpf et al. Proc Natl Acad Sci USA 96: 14007-12 (2000).
57. Huang et al., Mol Cell Endocrinol 159: 15-24 (2000).
58. Pelicano et al., Oncogene 15: 2349-59 (1997).
59. Nagpal et al., Invest Dermatol 106: 269-74 (1996).
60. Chu et al., J Nutr 129: 1846-54 (1999).
61. Chou et al., J Clin Endocrinol Metab 54: 1174-80 (1982).
62. Chou et al., In Vitro 19: 571-5 (1983).
63. Altucci et al., Nat Med 7: 680-6 (2001).
64. Bauvois et al., Oncogene 19: 265-72 (2000).
65. Rosewicz et al, FEBS Lett 368: 45-8 (1995).
66. Rosewicz et al., Gastroenterology 109: 1646-60 (1995).
67. Quelo et al, Biochem Biophys Res Commun 271: 481-91 (2000).
68. Tremblay et al, Biol Reprod 60: 541-5 (1999).
69. Kobayashi et al, Int J Hyperthermia 6: 785-92 (1990).
70. Kobayashi et al., Biochem Biophys Res Commun 238: 73843 (1997).
71. Niles et al, Cancer Invest 6: 3945 (1988).
72. Kaiser et al., Gastroenterology 113: 920-9 (1997).
73. Nakagawa et al., Differentiation 62: 249-57 (1998).
74. Reynolds et al., Cancer Lett 134: 53-60 (1998).
75. Ouellet et al., Biochim Biophys Acta 1048: 194-201 (1990).
76. Kwon et al, Dev Dyn 193: 193-8 (1992).
77. Gaetano et al, Eur J Cancer 4: 447-52 (1995).
78. Maden et al. Bioessays 21: 809-12 (1999).
79. Sakai et al., Genes Dev 15: 213-25 (2001).
80. Abu-Abed et al., Genes Dev 15: 22640 (2001).
81. Mira et al., J Cell Physiol 185: 302-9 (2000).
82. Rexer et al, Cancer Res 61: 7065-70 (2001).
83. Chetyrkin et al, J Biol Chem 276: 22278-86 (2001).
84. Nicke et al., Biochem Biophys Res Commun 261: 572-7 (1999).
85. Adachi et al., Tumour Biol 22: 247-53 (2001).
86. Lointier et al., Anticancer Res 12: 1327-30 (1992).
87. Cintron et al, Dis Colon Rectum 39: 406-9 (1996).
88. Duester et al, Eur J Biochem 267: 4315-24 (2000).
89. Ayer et al., Cell 72: 211-22 (1993).
90. Ayer et al. Genes Dev 7: 2110-9 (1993).
91. Hurlin et al., Cold Spring Harb Symp Quant Biol 59: 109-16 (1994).
92. Amati et al, Curr Opin Genet Dev 4: 102-8 (1994).
93. Chin et al., Proc Natl Acad Sci USA 92: 8488-92 (1995).
94. Ferguson et al., Neuroreport 12: A55-62 (2001).
95. Chawengsaksophak et al., Nature 386: 84-7 (1997).
96. Lorentz et al., J Cell Biol 139: 1553-65 (1997).
97. Beck et al., Proc Natl Acad Sci USA 96: 7318-23 (1999).
98. Tamai et al., Cancer Res 59: 2965-70 (1999).
99. Valcanis et al., Int J Dev Biol 41: 375-8 (1997).
100. Szeto et al., Cancer Res 61: 4197-205 (2001).
101. Easwaran et al., Curr Biol 9: 1415-8 (1999).

### EXAMPLE

Materials and Methods

Cell Culture and Drug Treatments. HT29, HCT116 and RKO colon adenocarcinoma cells were cultured as recommended by the American Type Culture Collection. HT29 APC-inducible and β-galactosidase-inducible cells were kindly provided by Dr. Bert Vogelstein (Johns Hopkins Oncology Center, Baltimore, Md.). For treatments with 5-Aza-CdR, cells were exposed to 5 μM 5-Aza-CdR (Sigma) at 24 hours after passage in complete culture medium. Control cultures were treated in parallel with vehicle (PBS). Forty-eight hours after drug addition, culture media was replaced with drug-free media. Control and 5-Aza-CdR treated cells were subcultured at equal densities three days after the initial treatment. In certain experiments, cells were exposed to a retinoic acid mixture (comprised of 1 μM all-trans retinoic acid, 1 μM 9-cis retinoic acid, and 1 μM 13-cis retinoic acid) or ethanol vehicle. Twenty-four hours prior to addition of retinoic acid or ethanol vehicle, normal media was replaced with media containing charcoal-stripped serum.

Microarray Analysis. Slides were produced using a Generation m Microarray Spotter (Molecular Dynamics). Each microarray contained 4608 minimally redundant cDNAs spotted in duplicate on 3-aminopropyl-trimethoxy silane (Sigma) coated slides and UV crosslinked in a Stratalinker (Stratagene). The cDNA clones on the microarray were obtained from Research Genetics and Genome Systems. Transformants were grown overnight at 37° C. in 96-well microtiter dishes containing 0.2 ml per well of TB supplemented with ampicillin. Cultures were transferred to a multiscreen 96-well glass fiber filtration plate (Millipore) and growth medium voided. Twenty-five μl of 25 mM Tris-HCl, pH 8; 10 mM EDTA, 50 μl of 0.2N NaOH, 1% SDS and 160 μl of 0.7M potassium acetate, pH 4.8; 5.3M guanidine hydrochloride were added to each well of the glass filtration plate. Cell lysates were drawn through the glass filters under vacuum and filter-bound DNA was washed four times with 200 μl of 80% ethanol. Plasmid DNAs were eluted by centrifugation following the addition of 65 μl of distilled H₂O. Samples were collected in a 96-well microtiter dish during centrifugation.

Generation of microarray probes, microarray hybridizations, and scanning Total RNA was isolated using Trizol reagent (Invitrogen) and poly-A RNA was selected using an Oligotex Kit (Qiagen). First-strand cDNA probes were generated by reverse transcription of one μg of purified mRNA with SuperScript II (Gibco) after the addition of Cy3-dCTP or Cy5-dCTP (Amersham Pharmacia). Following synthesis, RNA/cDNA hybrids were denatured and the mRNA was hydrolyzed with NaOH. For purification, the single-stranded cDNA probe was transferred to a Millipore glass-fiber filtration plate containing two volumes of 150 mM potassium acetate, pH 4.8, 5.3M guanidine hydrochloride. The mixture was voided by vacuum and bound cDNA washed four times with 80% ethanol. Probes were eluted by addition of 50 μl of distilled H₂O and were recovered by centrifugation. Next, the probes were reconstituted in 30 μl of 5×SSC, 0.1% SDS, 0.1 μg/ml salmon sperm DNA and 50% formamide. After denaturation at 94° C., the hybridization mix was deposited onto the slide under a cover slip.

Hybridizations were performed overnight at 42° C. in a humidified chamber. Following hybridization, slides were washed for 10 minutes in 1×SSC, 0.2% SDS and then for 20, minutes in 0.1×SSC, 0.2% SDS. Slides were dipped in distilled water, dried with compressed air and the fluorescent hybridization signatures were captured using the “Avalanche” dual laser confocal scanner (Molecular Dynamics). Fluorescent intensities were quantified using ArrayVision 4.0 (Imaging Research).

Transfections and Luciferase Assays. Transfection reagents included Fugene 6 (Roche Biochemicals) and Lipofectamine Plus (Invitrogen) for the transfection of HCT116 cells and RKO cells, respectively. Transfection procedures were performed as described by the manufacturers. Cells were seeded at a density of 100,000 cells per well in twenty-four well plates and transfected the next day. Transfections were performed using 0.6 μg DNA (including 0.06 μg normalization vector, 0.12 μg reporter vector, and 0.42 μg expression vector), and, in the absence of further treatment, cells were harvested twenty-four hours after the start of transfection. In certain experiments, media containing charcoal-stripped serum as well as either retinoic acid mixture (see above) or ethanol vehicle was added to cells after transfection, and cells were harvested twenty-four hours after media change. Luciferase values were analyzed using a Dual Luciferase Assay System (Promega). Transfection efficiencies were normalized by dividing the Firefly luciferase activity from each dish by the Renilla luciferase activity from the same dish. Data in each experiment are presented as the mean+/−S.D. of duplicates from a representative experiment. All experiments were performed at least three times producing qualitatively similar results.

Plasmids. Regions spanning −2228 to +1071 (in reference to translational start site) of the RDHL promoter and −1637 to +83 of the RDH5 promoter were PCR-amplified from normal human genomic DNA (Clontech). PCR products were then inserted behind the firefly luciferase gene in the pGL3basic vector (Promega) to create RDHL:LUC and RDH5:LUC, respectively. RDHL:LUC primers included a forward primer (5-GAAGATACACTTGGGTAGAAG-3) and a reverse primer (5-ACACCAGTTCCCATTCCTACTC-3). RDH5:LUC primers included a forward primer (5-GCTGCCTCCAGTCAGGTTAC-3) and a reverse primer (5-TTACCTCTCTGTGGCGAAAGC-3). PRL:LUC contains −36 to +36 of the prolactin gene and was kindly provided by Andrew Thorburn (Wake Forest University, Winston-Salem, N.C.). The CDX1 and CDX2 expression vectors were constructed by RT-PCR from normal colon RNA. The RT-PCR products were cloned into a pCDNA3.1 His C vector (Invitrogen). CDX1 primers included a forward primer (5-GCGCGGATCCATGTATGTGGGCTATGTGC-3) and a reverse primer (5-GCGCGAATTCCTATGGCAGAAACTCCTCT-3). CDX2 primers included a forward primer (5-GCGCGGATCCATGTACGTGAGCTACCTC-3) and a reverse primer (5-GCGCGAATTCTCACTGGGTGACGGTGG-3). For luciferase assays, RDH5:LUC or RDHL:LUC reporters were co-transfected with a Rous sarcoma virus (RSV)-Renilla luciferase reporter plasmid that was used to normalize transfection efficiencies.

Northern blotting. Total RNA was isolated using Trizol (Invitrogen) followed by poly-A RNA selection using a PolyA Tract mRNA Isolation kit (Promega). Poly-A RNA was fractionated through formaldehyde-containing agarose gels and transferred onto nylon membranes (Amersham Pharmacia). Probes were generated using the Rediprime II random prime labeling system (Amersham Pharmacia) supplemented with ³²P-dCTP. Hybridizations with ³²P-labeled probes were carried out using ULTRAhyb buffer (Ambion) as recommended by the manufacturer.

Results

Identifying Signaling Pathway Alterations: Retinoid Response Genes

Microarray expression analyses were carried out on colon polyps and colon tumors in comparison to normal. A striking feature of the colon tumor progression data was that approximately 80% of the differentially expressed genes were down in polyp and tumor tissues as compared to normal (data not shown). A profound absence of retinoic acid (RA) response genes in the polyp and tumor array data were observed (known RA response genes are indicated in bold, italic in Table 1). Of the most consistently down-regulated genes in colon polyps and tumors, nearly 25% are known targets of RA. The absence of RA response genes from colon polyps and tumors leads to an intriguing model explaining the lack of differentiation in colonocytes with mutated APC. Specifically, neoplastic colonocytes may be deficient in RA and/or incapable of responding to RA.

Retinol Dehydrogenases are Missing from Neoplastic Colon

The above data led the present inventors to investigate the basis for the absence of RA response genes in further detail. Attention was drawn to two additional genes displayed on the microarray overnight. Each of these genes was down-regulated in both polyp and tumor tissues relative to normal. Moreover, their absence was noted in over 70% of the neoplastic tissues examined (FIG. 1). As such, they closely paralleled the absence of RA response genes. The first gene encoded RDH5 (retinol dehydrogenase 5), an enzyme that catalyzes the conversion of retinol into retinal. The second gene encoded RDHL (retinol dehydrogenase-like), a recently described, novel retinol dehydrogenase [described by Soref et al (47), but referred to as hRDH-TBE]. The absence of RDH5 and RDHL from colon polyps and tumors presented a model explaining the lack of RA response genes. Specifically, neoplastic colonocytes may lack the ability to synthesize RA. Lack of RA response genes could reflect this lack of RA biosynthesis. In view of this, we decided to characterize RDH5 and RDHL more completely.

The tissue distribution of RDHL and RDH5 was analyzed by performing northern analyses on mRNAs from multiple human tissues. Hybridization with full-length RDHL identified a 1.9 kb mRNA species that was primarily expressed in the colon (FIG. 2). Although there are three potential splice variants of the RDHL gene (as deposited in Genbank by Accession clones AF067174, AF240698 and AF240697), RT-PCR confirmed that normal colon (data not shown) expresses the isoform corresponding to clone AF067174, the same splice variant characterized as a retinol dehydrogenase (47). Limited expression of RDHL was detected in heart, spleen, placenta and lung (FIG. 2). In contrast, a probe specific for RDH5 hybridized to a 1.4 kb mRNA species with the highest levels appearing in liver and kidney (FIG. 2). Limited expression of RDH5 was also observed in heart, skeletal muscle, colon and small intestine. Analysis of the blot with a probe specific for β-actin, indicated similar mRNA loading in each lane (FIG. 2) and confirmed the differential tissue expression of RDHL and RDH5. These data indicate that RDHL is highly specific to normal colon and raises the possibility that it serves as the primary source of retinol dehydrogenase activity in colon tissue. Furthermore, the particular expression patterns of RDHL and RDH5 indicate unique mechanisms controlling the expression of each gene in different tissues.

The expression of RDH5 and RDHL in patient matched normal and tumor tissues was then examined. A dot blot analysis of mRNAs from 11 colon tumors and 7 rectal tumors confirmed the observation that RDH5 and RDHL are lost in colorectal tumors (data not shown). Interestingly, a loss of RDH5 from kidney tumors also was found (data not shown).

1. A composition for treating a patient suffering from a colon tumor or tumor polyps, comprising an effective amount of a retinoid receptor agonist and a permissive factor therefor.

2. The composition according to claim 1, wherein said retinoid receptor agonist is retinoic acid or a derivative thereof.

3. The composition according to claim 2, wherein said permissive factor is a cdx2 molecule.

4. A host cell transfected with the vector according to claim 30.

5. A method for treating a patient suffering from a colon tumor, comprising the steps of administering to said patient an effective amount of the composition according to claim 1, and observing (i) a decreased size in the colon tumor in said patient, or (ii) a lack of increase in size of said colon tumor after predetermined amount of time.

6. The method according to claim 5, wherein said composition stimulates expression of a gene selected from the group consisting of RDHL, CEACAM-1 and CEACAM-5 in said patient.

7. The method according to claim 5, comprising administering to said patient a composition comprising an effective amount of a retinoid receptor agonist and a permissive factor therefore, wherein said retinoid receptor agonist is retinoic acid or a derivative thereof.

8. The method according to claim 7, wherein said permissive factor is a cdx2 molecule.

9. A method for treating a patient suffering from colon polyps, comprising the steps of administering to said patient an effective amount of a composition comprising a retinoid receptor agonist, and observing (i) a decreased size in the colon tumor in said patient, or (ii) lack of increase in size of said colon tumor after predetermined amount of time.

10. The method according to claim 9, wherein said composition stimulates expression of a gene selected from the group consisting of RDHL, CEACAM-1 and CEACAM-5 in said patient.

11. The method according to claim 9, wherein said composition further comprises a permissive factor for said retinoid receptor agonist.

12. The method according to claim 11, wherein said permissive factor is cdx2.

13. A kit, comprising (i) a plurality of host cells according to claim 4 and (ii) one or more test molecules.

14. A method for determining whether a test molecule can upregulate RDHL expression in a cell comprising the steps of: administering said test molecule to a cell according to claim 4; and measuring the level of RDHL enzymatic activity, wherein an increase in RDHL activity is indicative that said test molecule upregulates RDHL expression.

15. The method according to claim 14, further comprising administering a retinoid receptor agonist to said cell, and wherein said test molecule is a permissive factor therefor.

16. The method according to claim 15, wherein said retinoid receptor agonist is retinoic acid or a derivative thereof.

17. The method according to claim 14, wherein said test molecule is not a retinoid receptor agonist.

18. A method for determining whether a test molecule can upregulate RDHL, CEACAM-1 or CEACAM-5 expression in a cell comprising the steps of: administering said test molecule to a cell according to claim 4; and measuring the level of gene expression of said RDHL, CEACAM-1 or CEACAM-5, wherein an increase in gene expression is indicative that said test molecule upregulates said RDHL, CEACAM-1 or CEACAM-5.

19. The method according to claim 18, wherein said measuring step involves determining the level of messenger RNA corresponding to said RDHL, CEACAM-1 or CEACAM-5.

20. The method according to claim 18, wherein said measuring step involves determining the level of protein encoded by said RDHL, CEACAM-1 or CEACAM-5 gene.

21. The host cell according to claim 4, wherein said host cell is derived from a vertebrate.

22. The host cell according to claim 4, wherein said RDHL promoter is operably linked to a reporter molecule.

23. A method for preventing colon polyp formation in a patient, comprising the steps of: identifying a patient having a predisposition to colon polyp formation, and administering to said patient an effective amount of the composition according to claim 1.

24. The method according to claim 23, wherein said composition stimulates expression of a gene selected from the group consisting of RDHL, CEACAM-1 and CEACAM-5 in said patient.

25. A method for preventing colon polyp formation in a patient, comprising the steps of: identifying a patient having a predisposition to colon polyp formation, and administering to said patient an effective amount of a composition comprising a retinoid receptor antagonist.

26. The method according to claim 25, wherein said composition stimulates expression of a gene selected from the group consisting of RDHL, CEACAM-1 and CEACAM-5 in said patient.

27. A method for preventing colon tumor formation in a patient, comprising the steps of: identifying a patient having colon polyps or a predisposition to colon tumor formation, and administering to said patient an effective amount of the composition according to claim 1, and observing (i) a decreased amount of colon polyps in said patient, or (ii) lack of increase in the amount of colon polyps in said patient after predetermined amount of time.

28. The method according to claim 27, wherein said composition stimulates expression of a gene selected from the group consisting of RDHL, CEACAM-1 and CEACAM-5 in said patient.

29. An isolated DNA molecule comprising an RDHL promoter.

30. A vector comprising the DNA molecule according to claim 29.


SEQUENCE LISTING (10 sequences)

SEQ ID NO: 1 - DNA, 2519 bp, Homo sapiens
ccagaaggcgcccaaagcctgagtcacagccggtgatgaagacaaaggcattgctggcgggcaggctctgccggtccctgagcaaccacagcactgcccagagtaaggcacccagcagaagaggcagccacatagctggagcccaagtgacctacaggtggcagcctaggctgggcagaaaaacttgtccagtttactgtggccctcaagccctttcccctaataccctccctagctggaagcatctggtgtgagcatattgagaaattatctggttggcagataattggccaaccatggctcatctgttcaaagcaaagatgtgggcagaccctgtatttgtggctaacttaactaaaaccacatgagccagatcctgtgtacagacctgctgtatggagcagaggcatctttttttttttttttttttttttgagatggagtctcactctgtcgcccaggctggagtgcagcagaggcatcttcttgcttgcctcacaccacctctccttctaaaccagggacactgaaccttctatatgcaacctgggtcttgtgtttggaatgatgtatagggccaagaggtttgctgtgtgatactgagcatctcactgtctctctcaacctattttctcatctaccaagtggggctaagacccaccaggtccacctcagagttgtggaggatcaaacggggataatgtgtgaggaagcatgttaaaagctgaaagttttgagttggcaatacatgtaatatctggctagtcttctcagttcccccaggctccctagtatccacttacctctctgtggcgaaagctggcaggtactacagcgcccgaagcttattcttcggcagggtttaagagactgctcccagtcttcccagcctcccacactaactactgctaaggccaaagtccggagtgggtggacttggatgccaggcgtattaagtgtatgtgtgacagggtggggataggactgaggaggaggaggagccagggaggctggagcttgcctggctgaagccaggacttggcagaggccagatttgcctaggctccccccaccccttggctgtctttctcagcctgggttcctgaccatctatgtcatggtctccaaggaggagggagctgtcaaggatggcagctgcactgctctgttggtaacggttactggacctgtcattcctcccccttctcagccctgaagtgcccccaggaattaatctcccacctggctcagactcatgacactgcttagagttagcctgggagaggccatgagcacgtggagaggcagaaagccagagctggggaccaggcctgtaccttgccttgtttctcttctgtaggaaacaagtcctgaattaggaagcttggggcagggctactaggaccttctctcaggagggatgggaactcccaccctctgcttgcctaacagacacctgcacaagggccatggaggagaagagagttgggatagtaagaggaagctagttccaccctctccccaaaaggtacctgtccctcagggaaaccacctttgaaccttccactcccacccccgacactggagctagtagctctctgaaacacaccacagagaaacggaggcttgtgttttattcaaggcttgtcagcctccctccttccccctgcgggtgaggtaggagggatagggttgggggagggaacaggggctaggaaggggcagactgcagctgccctttgtagacatattccagtgcagtggcaatggtgcgcatgtccagctcgatgctccgagcccagttctccacatccccaatttcctgggaagggggagaggaggtgggagcccaggaatcagaatgggtagagtatggcttccagtctggggaatacgttttattcatctcctggaaaaggctttacttggacccaccaactgcttttctttctttctctaccttttctgacactggggctatagatggggtttctgggaccaccctacttcctccttaacctaaacctgaggtaacctgactggaggcagccaatgggggcccaggattggggtggtgaggtagggagtatggcccaccttgagtgcctggttgaagttctccaccattccgatccactggcctgtctgcttggcaaattgggcagcctggacctgtagggtcttcacctcatggtccagctttctctggttcatgtaggcctgggccacactggggggttggaggagggaggagatcagctccagctagttgggggtggtctccgggactaatattccattgcagcagtgggagttcagccaagaatcagggctcataattttggccatttttggatgtgatgcaaatgactaccagtacatgcaaataaacccagctccttcctttcactcccaggattcccaggagagccaagctcggagctgactagaaacataccagctccaaaccccaaaaaggagttcctgcagctgagtgggggaagggagggaaaacctgg

SEQ ID NO: 2 - DNA, 1251 bp, Homo sapiens
ttacctctctgtggcgaaagctggcaggtactacagcgcccgaagcttattcttcggcagggtttaagagactgctcccagtcttcccagcctcccacactaactactgctaaggccaaagtccggagtgggtggacttggatgccaggcgtattaagtgtatgtgtgacagggtggggataggactgaggaggaggaggagccagggaggctggagcttgcctggctgaagccaggacttggcagaggccagatttgcctaggctccccccaccccttggctgtctttctcagcctgggttcctgaccatctatgtcatggtctccaaggaggagggagctgtcaaggatggcagctgcactgctctgttggtaacggttactggacctgtcattcctcccccttctcagccctgaagtgcccccaggaattaatctcccacctggctcagactcatgacactgcttagagttagcctgggagaggccatgagcacgtggagaggcagaaagccagagctggggaccaggcctgtaccttgccttgtttctcttctgtaggaaacaagtcctgaattaggaagcttggggcagggctactaggaccttctctcaggagggatgggaactcccaccctctgcttgcctaacagacacctgcacaagggccatggaggagaagagagttgggatagtaagaggaagctagttccaccctctccccaaaaggtacctgtccctcagggaaaccacctttgaaccttccactcccacccccgacactggagctagtagctctctgaaacacaccacagagaaacggaggcttgtgttttattcaaggcttgtcagcctccctccttccccctgcgggtgaggtaggagggatagggttgggggagggaacaggggctaggaaggggcagactgcagctgccctttgtagacatattccagtgcagtggcaatggtgcgcatgtccagctcgatgctccgagcccagttctccacatccccaatttcctgggaagggggagaggaggtgggagcccaggaatcagaatgggtagagtatggcttccagtctggggaatacgttttattcatctcctggaaaaggctttacttggacccaccaactgcttttctttctttctctaccttttctgacactggggctatagatggggtttctgggaccaccctacttcctccttaacctaaacctgaggtaacctgactggaggcagc

SEQ ID NO: 3 - DNA, 21 bp, Artificial Sequence
  Description of Artificial Sequence Synthetic primer
gaagatacacttgggtagaag

SEQ ID NO: 4 - DNA, 23 bp, Artificial Sequence
  Description of Artificial Sequence Synthetic primer
acaccagttcccatttcctactc

SEQ ID NO: 5 - DNA, 20 bp, Artificial Sequence
  Description of Artificial Sequence Synthetic primer
gctgcctccagtcaggttac

SEQ ID NO: 6 - DNA, 21 bp, Artificial Sequence
  Description of Artificial Sequence Synthetic primer
ttacctctctgtggcgaaagc

SEQ ID NO: 7 - DNA, 29 bp, Artificial Sequence
  Description of Artificial Sequence Synthetic primer
gcgcggatccatgtatgtgggctatgtgc

SEQ ID NO: 8 - DNA, 29 bp, Artificial Sequence
  Description of Artificial Sequence Synthetic primer
gcgcgaattcctatggcagaaactcctct

SEQ ID NO: 9 - DNA, 28 bp, Artificial Sequence
  Description of Artificial Sequence Synthetic primer
gcgcggatccatgtacgtgagctacctc

SEQ ID NO: 10 - DNA, 27 bp, Artificial Sequence
  Description of Artificial Sequence Synthetic primer
gcgcgaattctcactgggtgacggtgg